(19) United States
(12) Patent Application Publication
(10) Pub. No.: US 2006/0155269 A1
(43) Pub. Date: Jul. 13, 2006

(54) Epicardial ablation using focused ultrasound

(75) Inventor: Reinhard Warnking, Setauket, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: ProRhythm, Inc., Ronkonkoma, NY (US)

(21) Appl. No.: 11/324,542

(22) Filed: Jan. 3, 2006

Related U.S. Application Data
(60) Provisional application No. 60/643,281, filed on Jan. 12, 2005.

(51) Int. Cl. A61B 18/04

(52) U.S. Cl. 606/27

(57) ABSTRACT

Apparatus for treating cardiac arrhythmias includes an ultrasonic ablation device incorporating an ultrasonic emitter (22, 400), means (10, 14, 402, 404) for positioning the ablation device outside of the heart but adjacent the epicardial surface of the heart, and means (26, 28, 30) for focusing the ultrasonic energy emitted by the emitter into an ablation region, so that the ablation region is disposed within the wall of the heart.

### CROSS-REFERENCE TO RELATED APPLICATIONS

The present application claims the benefit of the filing date of U.S. Provisional Patent Application No. 60/643,281, filed Jan. 12, 2005, the disclosure of which is hereby incorporated by reference herein.

### BACKGROUND OF THE INVENTION

The present invention relates to apparatus and methods for cardiac ablation.

The repetitive contraction of the heart is controlled by electrical signals propagating within the tissue of the heart itself. In a normal heart, these signals travel along well-defined pathways in a controlled manner, so that the various portions of the heart muscle contract in a well-defined rhythm. In diseased states referred to as cardiac arrhythmias, the normal pattern of conduction is disrupted, typically by the presence of abnormal signal sources, conductive pathways or both, which cause abnormal signals to propagate within portions of the heart. For example, in atrial fibrillation, abnormal signals conducted along abnormal pathways in the vicinity of the pulmonary veins cause undesired contraction of the muscle constituting the left atrium out of synchronization with the remainder of the cardiac cycle. Arrhythmias decrease the pumping efficiency of the heart, and can cause thrombogenesis or clotting of the blood. In some cases, arrhythmias can be controlled by medication. In other cases, however, arrhythmias must be treated by physically disrupting the abnormal conductive pathways. This can be done by an open heart surgical procedure known as a maze procedure, in which the surgeon mechanically cuts the heart wall along a path which intercepts the abnormal conductive pathways. The scar tissue which forms along the cut lines does not conduct electrical impulses, and thus blocks conduction along the abnormal conductive pathways. In the case of atrial fibrillation, the abnormal conductive paths extend from within the pulmonary veins into the surrounding tissue, and accordingly, scars must be formed in the vicinity of the pulmonary veins, typically as one or more complete or partial loops extending around the openings or ostia of the pulmonary veins.

Considerable effort has been devoted to development of catheter-based procedures to replace the conventional open heart surgical procedures. Such catheter-based procedures involve threading a catheter through the circulatory system into the heart and introducing an ablation device into the interior of the heart. For example, in treatment of atrial fibrillation, an ablation device typically is threaded into the heart through an introducer sheath extending through the right atrium and through the septum at the fossa ovalis, into the left atrium. The ablation device may be arranged to apply essentially any treatment which can cause conversion of the myocardial tissue to scar tissue. For example, it has been proposed to apply radiofrequency (RF) energy; light energy, such as light from a laser; and heat energy. Other types of ablation devices apply extreme cold using a cryogenic agent to freeze the tissue. Still other types of ablation devices are arranged to introduce a chemical agent into the tissue. To provide effective treatment, the lesions should be “transmural,” i.e., the lesions should extend substantially through the thickness of the heart wall.

Many ablation devices operate only at a single point, so that the physician can create a linear or loop-like lesion by tracing along the desired path on the interior of the heart with the ablation device, while periodically or continuously actuating the ablation device to apply energy. This approach requires extraordinary manipulation by the physician, and also requires that the probe carrying the ablation device be arranged to allow controlled manipulation within the heart.

Other proposals have been advanced for providing loop-like or linear ablation devices which are arranged to apply energy at numerous points or continuously along a predetermined length of the probe, so that the physician can position the probe with the probe extending along the path of the desired lesion and then actuate the probe to form the entire lesion or a substantial part of the lesion without having to trace the lesion with the tip of the probe. Typically, these proposals have been directed to probes having radiofrequency ablation electrodes disposed at spaced locations along their length. Radiofrequency ablation has significant drawbacks, including scarring of the delicate lining of the heart leading to thrombogenesis. Suggestions have been advanced that ultrasonic energy could be substituted for radiofrequency energy in such a system. However, simple ultrasonic transducers of a size which can be practically mounted on such a probe can provide only a very limited power density in the tissue being heated. Therefore, considerable time is required to heat the particular tissue to be ablated. This, in turn, leads to substantial conduction of heat to neighboring tissues, making it difficult or impossible to heat tissue within the wall of the heart without also damaging surrounding tissues.

As disclosed, for example, in certain embodiments of U.S. Published Patent Application No. 2002/0176757, published Sep. 9, 2004, now U.S. Pat. No. 6,749,378; and U.S. Published Patent Application No. 2002/0065512, published May 30, 2002, now U.S. Pat. No. 6,635,054, the disclosures of which are incorporated by reference herein, ultrasonic energy can be applied to the interior of the heart using a unique inflatable reflector structure which directs the energy from a cylindrical transducer onto a loop-like path surrounding the axis of the cylindrical transducer, and which also focuses the energy from the transducer into a small ablation region extending along this loop-like path, so that the energy from the transducer is concentrated within this ablation region. Systems of this nature have been successfully employed to treat atrial fibrillation.

Another approach which has been suggested is to ablate the myocardial tissue from the epicardial surface of the heart, i.e., from outside of the heart, by introducing an ablation device into the epicardial space, within the epicardial sack surrounding the heart. For example, as shown in U.S. Pat. No. 6,161,543, an elongated probe is introduced into the patient's chest through an opening in the chest wall and through the pericardium into the pericardial space. The probe is curved and positioned so that the curved probe extends along a portion of a loop-like path encircling the pulmonary veins. Once the probe is placed, it is filled with a cryogenic fluid, and thus ablates the myocardial tissue from outside of the heart by freezing the tissue in contact with the probe. Although the '543 patent mentions that the ablation probe can use RF, ultrasound, microwave, laser, heat, chemical agents, biological agents and light-activated agents, it does not provide a practical way of ablating the myocardial tissue from outside of the heart using ultrasound. Likewise, U.S. Pat. No. 6,579,285 discloses a generally similar procedure using a curved probe to apply infrared radiation or microwave radiation along a curved path along the epicardial surface, and thereby form a lesion in the heart wall. The '285 patent also mentions briefly that “other ablation means, such as RF heating, cryogenic cooling, ultrasound, microwave, ablative fluid injection and the like” can be used, but provides no practical way of forming a lesion using ultrasonic energy.

Another type of device which has been proposed for ablating the heart wall from the epicardial surface includes a forceps-like device having two pivotally connected halves, with each half having a curved portion resembling a blade of a forceps. Ablation elements are disposed along these curved portions so that the device can be inserted into the chest cavity and positioned around the pulmonary vein, so that the ablation elements carried on the curved blades are disposed along arcuate paths on the epicardial surface of the heart. The '767 patent mentions that the ablation elements may be RF, ultrasound, laser or cryogenic elements, but here again, fails to provide any practical way of applying ultrasonic energy to produce a transmural lesion.

Despite all of these efforts in the art, still further improvement would be desirable. In particular, it would be desirable to provide apparatus and methods which can effectively form transmural lesions by ablation from the epicardial surface, without the drawbacks associated with RF, cryoablation, microwave and optical ablation.

### SUMMARY OF THE INVENTION

One aspect of the present invention provides apparatus for treating cardiac arrhythmias. The apparatus according to this aspect of the invention desirably includes an ultrasonic ablation device including an ultrasonic emitter and means for focusing ultrasonic waves emitted by the emitter into an ablation region. As used in this disclosure, the term “focusing” refers to the process of concentrating the power delivered by ultrasonic waves by directing the ultrasonic waves along at least a portion of their path of travel from the emitter to the ablation region. The waves converge with one another. Stated another way, in a focusing system, the ultrasonic power density per unit area increases with distance from the emitter along at least a portion of the path from the emitter to the ablation region.

The apparatus according to this aspect of the invention desirably includes means for positioning the ablation device outside of the heart adjacent the epicardial surface of the heart, so that the ablation region is disposed within the wall of the heart.

Preferably, the means for positioning includes an elongated probe having a lengthwise direction, and the ultrasonic ablation device is carried by the probe. The means for focusing desirably is operative to focus the energy from the emitter into an elongated ablation region extending generally parallel to the lengthwise direction of the probe, but offset from the probe in a lateral direction transverse to the lengthwise direction. Preferably, the emitter includes one or more ultrasonic emitting elements mounted to the probe, and the one or emitting elements define an emitting surface extending in the lengthwise direction along the probe. The focusing means may include a lens carried on the probe, which desirably is also elongated in the lengthwise direction. Such a lens may include an inflatable lens or a Fresnel lens overlying the emitting surface. Alternatively or additionally, the focusing means may include one or more structures carried by the probe defining a pair of reflective surfaces which, in turn, form the boundary of a slot extending lengthwise along the probe. The slot has an entry opening adjacent the emitting surface and an exit opening remote from the emitting surface. The reflective surfaces converge with one another from the entry opening to the exit opening, so that ultrasonic waves are directed, by repeated reflection from the converging surfaces, into the small exit opening.

In a further variant, the focusing means may include an elongated reflector extending along the probe, such reflector having surface regions which are positioned on the probe so that vectors normal to these surface regions converge with one another. For example, the reflector surface may include surface regions disposed on opposite sides of a medial plane extending in the lateral direction, and the surface regions may include regions disposed on opposite sides of the medial plane, these regions having normal vectors sloping toward the medial plane. In yet another variant, the emitting surface itself may include surface regions having normal vectors which converge with one another, and the focusing means may include the surface regions of the emitting surface itself and the elements which hold such emitting surface regions in position relative to one another.

The elongated probe may be an elongated rod-like or tubular structure, such as a catheter, which is preformed to a desired curved shape, which can be steered to a desired shape, or which can be bent by the physician to a selected shape prior to placement and retain such shape, or which may be adapted to assume a desired shape upon exposure to body temperature. Alternatively, the elongated probe may be a probe which can be selectively deformed by the physician during placement. For example, the probe may incorporate internal steering mechanisms which assist in forming the probe into a desired curved shape, or may be simply a flexible element which can be selectively deformed by external manipulation using other instruments. The means for positioning the emitting device may also include elements for introducing the probe, such as trocars or introducer sheaths arranged to be threaded into the chest cavity, and other tools which can be used to place the probe as desired. In a further variant, the elongated probe may be a resilient structure which is constrained by the introducer structure during placement, which returns to a desired shape upon release of such constraint.

In an alternate embodiment, the elongated probe may include a blade of a forceps-like device, so that the ultrasonic emitting element and focusing means are arranged along the length of such blade. The positioning means may further include an opposing element which may optionally bear another ultrasonic emitting device and focusing means. Thus, in this arrangement, the positioning means includes a forceps-like device.

These and other objects, features and advantages of the present invention will be more readily apparent from the detailed description of the preferred embodiments set forth below, taken in conjunction with the accompanying drawings.

### BRIEF DESCRIPTION OF THE DRAWINGS

FIG. 1 is a diagrammatic perspective view of apparatus according to one embodiment of the invention in conjunction with anatomical features of a patient.

FIG. 2 is a diagrammatic sectional view along line 2-2 in FIG. 1.

FIG. 3 is a diagrammatic sectional view depicting apparatus according to a further embodiment of the invention.

FIG. 4 is a diagrammatic sectional view depicting apparatus according to yet another embodiment of the invention.

FIG. 5 is a diagrammatic sectional view depicting apparatus according to a still further embodiment of the invention.

FIG. 6 is a diagrammatic sectional view depicting apparatus according to yet another embodiment of the invention.

FIG. 7 is a diagrammatic top plan view depicting apparatus according to a still further embodiment of the invention in conjunction with certain anatomical features.

### DETAILED DESCRIPTION

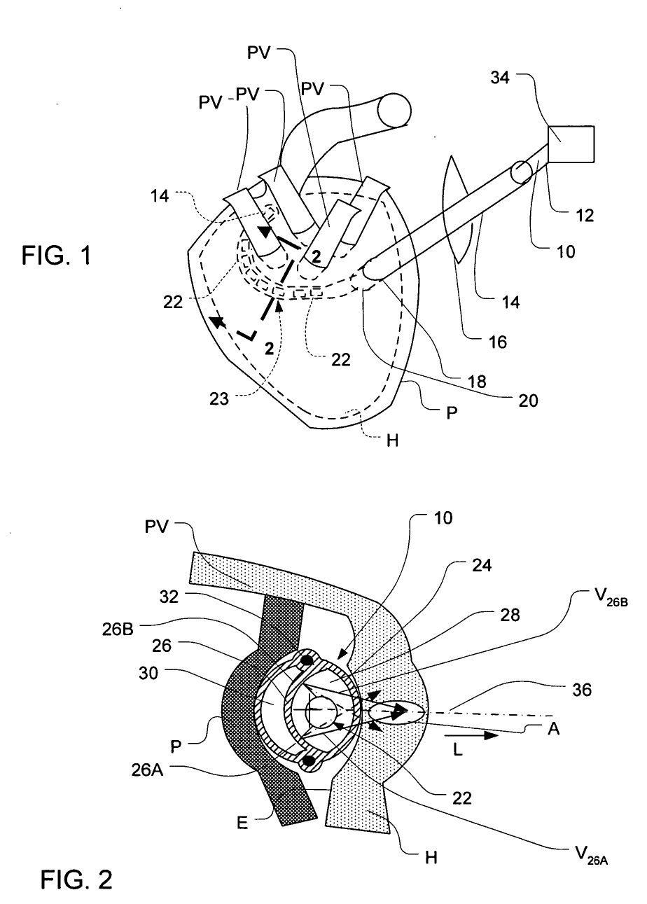

As seen in FIG. 1, apparatus in accordance with one embodiment of the present invention incorporates an elongated rod-like probe 10 having a proximal end 12 and a distal end 14, and having a lengthwise direction extending between these ends. The apparatus also includes a hollow tubular insertion guide 14 which may be a simple sheath or a trocar. Introducer 14 is dimensioned and constructed so that it can be inserted into the patient's body through a subxiphoid insertion into the pericardial sack P, so as to position the distal end 20 of the introducer 14 inside the pericardial sack. In FIG. 1, the structures enclosed by the pericardial sack are indicated in broken lines for clarity of illustration. Also, FIG. 1 depicts the pericardial sack P and other structures of the patient's heart H in an entirely schematic form.

Probe 10 has a large number of individual ultrasonic emitting elements 22 disposed along the length of the probe within an emitting region 23 near the distal end 14 of the probe, so that the various emitting elements together cooperatively constitute an elongated emitting element extending in the lengthwise direction of the probe, i.e., the direction along the probe towards and away from the distal end 14 of the probe. As best seen in FIG. 2, each emitting element 22 is a generally cylindrical element adapted to radiate ultrasonic energy from its exterior surface. The individual emitting elements 22 may be piezoelectric elements generally of the type described in U.S. Pat. No. 6,763,722, the disclosure of which is hereby incorporated by reference herein. As further discussed in the '722 patent, such an emitting element may include a hollow cylindrical piezoelectric element with structures arranged to pass cooling water inside the hollow element. Even very small elements of this general type can provide substantial radiated ultrasonic power as, for example, on the order of 50-100 watts from a transducer having a diameter on the order of 3 mm and a length on the order of 1 cm.

As also shown in FIG. 2, probe 10 incorporates a hollow body 24 having an interior wall 26 subdividing the interior space within the body into a first space 28 housing the emitters 22, and a second space 30. FIG. 2 shows the probe in sectional view as seen along cutting plane 2-2 (FIG. 1), such cutting plane being across the lengthwise direction of the probe, so that the lengthwise direction extends into and out of the plane of the drawing in FIG. 2. Thus, the features shown in FIG. 2 extend lengthwise along the probe at least in the emitting region 23 adjacent the distal end of the probe. The probe further includes pre-formed resilient wires 32 embedded in the wall of the probe body. Wires 32 are arranged to resiliently bias the emitting region 23 into the curved shape depicted in FIG. 1. Probe body 10 has a connector 34 schematically depicted in FIG. 1 at the proximal end 12 of the probe body. The probe body has lumens (not shown) extending longitudinally from the proximal end to the emitting region 23 and communicating with the first space 28 and second space 30. The connector 34 is arranged to connect the first space 28 to a source of a liquid such as a saline solution, and to connect the second space 30 to a source of a gas such as carbon dioxide.

In operation, after positioning introducer 14 with its distal end 20 extending through the pericardium P, the physician threads probe 10 into the pericardial space through the interior of the introducer and positions the probe with the emitting region in the position indicated in FIG. 1. In this condition, as seen in FIG. 2, the probe body is disposed between the pericardium P and the heart H, with the first space 28 facing toward the heart wall and confronting the epicardial or exterior surface E of the heart wall. The first space 28 is filled with an aqueous liquid, whereas the second space 30 is filled with a gas. Upon actuation of transducer elements 22, some of the ultrasound waves emitted by each transducer pass laterally in the direction indicated by arrow L in FIG. 2 and, thus, pass directly into the heart wall. Other ultrasonic waves pass in other directions transverse to the longitudinal direction (in the plane of the drawing in FIG. 2) and encounter the bounding wall 26 separating the first and second spaces. Due to the difference in acoustic impedance between the liquid in first space 28 and the gas in second space 30, bounding wall 26 acts as an almost perfect reflector for ultrasound. Desirably, bounding wall 26 is a relatively thin membrane, so that the reflective properties at the bounding wall are dominated by the properties of the liquid in space 28 and the gas in space 30, rather than by the material of the bounding wall. Bounding wall 26 has portions facing inwardly, towards a medial plane 36 extending in the lateral direction L. Stated another way, a vector V26A from a portion 26A of the boundary or reflector surface 26 normal to the surface in this region points inwardly toward medial plane 36, whereas vector V26B from a surface region 26B on the opposite side of the medial plane 36 slopes in the opposite direction so that it also slopes toward the medial plane in the lateral direction L. As will be appreciated from FIG. 2, surface 26, as see in section in a plane transverse to the lengthwise direction of the probe, is a concave reflector facing in the lateral direction L toward the heart H. The reflecting surface 26 reflects waves from emitting element 22 along mutually converging paths, sloping inwardly toward medial plane 36 in the lateral direction L. The converging ultrasonic waves are focused into an ablation region A extending along the medial plane 36, but offset in the lateral direction L from the probe body 24. With the probe positioned in the manner discussed above, ablation region A lies within the heart wall H. The focused ultrasonic waves rapidly heat the tissue within the ablation region and, thus, ablate the tissue in this region. The same process occurs at other points along the length of emitting region 23, so that the reflector surface 26 focuses energy from all of the various emitting elements 22 into an elongated ablation region A extending parallel to the lengthwise direction of the probe within the emitter region 23, such ablation region being disposed within the heart wall. The ablation, thus, forms an elongated lesion.

In the position indicated, the emitting region 23 is in the form of an arc extending partially around the pulmonary veins, and hence, the region will extend partially around the area occupied by the ostia of the pulmonary veins PV. One wall of one pulmonary vein is seen in FIG. 2 extending through the pericardium. After ablation, the same probe can be repositioned to form a further arc extending over a different portion of the heart wall, or replaced by a new probe using the same opening in the pericardium or a different opening. In this way, a complete lesion can be formed to entirely encircle the region occupied by the pulmonary veins. Other lesions can be formed at locations as desired.

In the method discussed above, the focused ultrasonic energy is directed inwardly, into the heart from the epicardial surface. This is highly advantageous. Some ultrasonic energy typically will pass through the wall of the heart. Because the ultrasonic energy is directed inwardly, any energy passing through the heart wall will be directed into the blood within the heart itself as, for example, the blood in the left atrium. Ultrasonic energy passing beyond the ablation region A in the lateral direction L (to the right as seen in FIG. 2) will diverge and spread outwardly from the medial plane 3C. Moreover, ultrasonic energy is only weakly absorbed by the blood. Therefore, any stray ultrasonic energy will be dissipated harmlessly, without causing substantial undesired heating of structures other than the particular ablation region A. Structures outside of the pericardium P will not be heated to any appreciable degree. Therefore, there is little or no chance of ablating anatomical structures located near the heart, such as nerves or the esophagus. The focused ultrasound in the ablation region provides rapid heating, which tends to minimize heat loss by conduction. This facilitates formation of a transmural lesion and minimizes thermal damage to adjacent portions of the heart wall and other tissues.

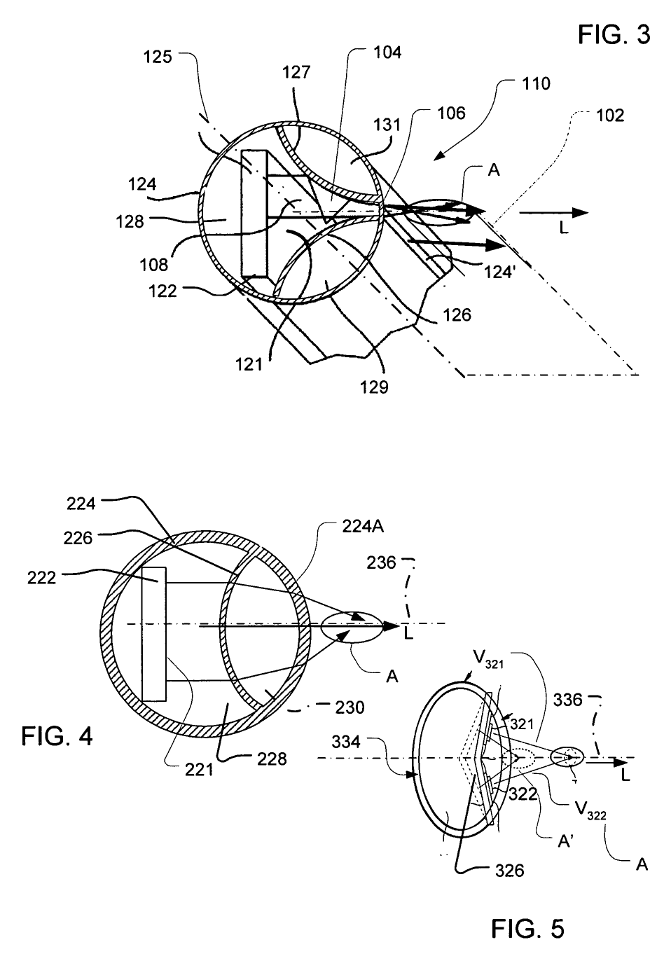

A probe 110 according to a further embodiment, partially shown in FIG. 3, includes a probe body which, again, is in the form of an elongated tubular body 124 having a lengthwise or axial direction indicated by axis 125 in FIG. 3. Probe body 124 includes a pair of arcuate boundary walls 126 and 127 which extend lengthwise along the probe, and which also extend in the lateral direction L, transverse to the lengthwise axis 125, so that these boundary walls lie on opposite sides of medial plane 102. As seen in cross-section in FIG. 3, walls 126 and 127 are disposed on opposite sides of a medial plane 102 and converge toward medial plane 102 so as to define a slot 104 encompassing the medial plane. The slot has a relatively narrow outlet opening 106 at the exterior surface of the probe body and a relatively wide entry opening 108 remote from the exterior surface of the probe body. Walls 126 and 127, and hence, slot 104 and exit opening 106, extend lengthwise along the probe body. The exit opening 106 of the slot is covered by a portion 124′ of the exterior bounding wall of the probe body. Walls 126 and 127 effectively subdivide the space inside of the probe body 124 into a first chamber 128, which includes the space within slot 104 inside the probe body, and two additional chambers 129 and 131 bounded by walls 126 and 127. An elongated ultrasonic transducer 122 is disposed in the first space. The ultrasonic transducer may include one or more elements extending along the lengthwise direction of the probe. The emitter in this case is substantially flat and has an emitting surface facing in the lateral direction L and confronting the entry opening 108 of slot 104.

The probe according to this embodiment of the invention is also arranged for connection to a source of a liquid such as a saline solution to fill the first chamber 128 and a source of a gas such as carbon dioxide to fill the second chambers 129 and 131. Here again, the differences in acoustic impedance between the liquid and the gas provide highly reflective interfaces at the converging walls 126 and 127 bounding slot 104. Thus, the slot is bounded by converging surfaces which are highly reflective to ultrasound. The apparatus according to this embodiment of the invention may include other features generally similar to those discussed above with references to FIGS. 1 and 2. Here again, the probe is positioned around the exterior of the heart, inside the pericardium, with the probe extending along the path of the lesion to be formed and with the lateral direction L of the probe facing inwardly, toward the epicardial surface of the heart. When the transducer 122 is actuated to supply ultrasonic waves, the waves encounter the converging reflective surfaces 126 and 127 and are repeatedly reflected from these surfaces so that they are effectively funneled to and through the narrow exit opening 106 of the slot. The focused waves exit through the wall portion 124′ at the exit opening. Thus, a highly concentrated beam of ultrasonic energy is directed into the ablation region A, just outside of the slot exit opening. Here again, the ablation region is an elongated region laterally offset from the probe body and extends along the path of the lesion to be formed. In the same manner as discussed above, when the probe body is in position relative to the heart, the ablation region lies within the myocardial tissue of the heart.

A probe according to a further embodiment of the invention (FIG. 4) includes an elongated ultrasonic emitter 222 having an emitting surface 221 facing in the lateral direction. The probe body 224 has an interior wall 226 which subdivides the space within the probe body into a first chamber 228 housing the emitter and a second chamber 230. In this embodiment, chambers 228 and 230 are filled with liquids having substantially the same acoustic impedance, but having different acoustic velocities. Thus, the interface at wall 226 is refractive rather than reflective. Desirably, first chamber 228 is filled with saline or other aqueous solution having acoustic impedance and acoustic velocity similar to that of bodily fluids, whereas chamber 230 is filled with a fluid having a lower acoustic velocity as, for example, a fluorocarbon. Thus, the refractive surface 226 tends to bend acoustic waves traveling in the lateral direction toward the medial plane 236. Similarly, the interface at wall portion 224A of the probe also forms a refractive interface, due to the difference in acoustic velocity between the medium in chamber 230 and the bodily fluids outside of the probe body. This refractive interface also tends to bend acoustic waves towards the medial plane 236.

As in the embodiments discussed above, the features seen in sectional view extend lengthwise along the probe body (into and out of the plane of the drawing in FIG. 4), and hence, extend along the emitting region of the probe. In this embodiment as well, ultrasonic waves are focused into an ablation region A adjacent the medial plane and laterally offset from the probe body.

In apparatus according to yet another embodiment of the invention (FIG. 5), the ultrasonic emitter includes two rows of emitting elements 321 and 322. Elements 321 and 322 face generally in the lateral direction L of the probe but lie on opposite sides of the medial plane 336. Element 321 defines a portion of the emitting surface having a normal vector V₃₂₁ sloping toward the medial plane 336 in the lateral direction, whereas element 322 defines a further emitting surface portion having a normal vector V₃₂₂ sloping in the opposite direction, so that the normal vectors converge toward the medial plane in the lateral direction L. In other respects, the embodiment of FIG. 4 is similar to the embodiments discussed above. In operation, ultrasonic waves emitted by elements 321 and 322 converge with one another in an ablation region A disposed along the medial plane, and thus, provide convergent, focused ultrasound within the ablation region.

As seen in FIG. 5, emitting elements 321 and 322 are mounted on portions of a wall 326, which subdivides the interior space within probe body 334. In a variant of the approach seen in FIG. 5, wall 326 may be deformed by differential fluid pressure on opposite sides of the wall so as to bend the wall into a more severe v-shape, as seen in broken lines in FIG. 5 at 326′, and thereby change the slopes of the normal vectors, as also shown in broken lines, so as to move the ablation region A to a new location A′, closer to the probe body. Here again, the configuration of the emitting elements seen in FIG. 5 extends lengthwise along the probe body. The emitting elements 321 and 322 may be formed as portions of a flexible, multi-element probe array, as disclosed in certain embodiments of U.S. Pat. No. 6,605,084, the disclosure of which is hereby incorporated by reference herein.

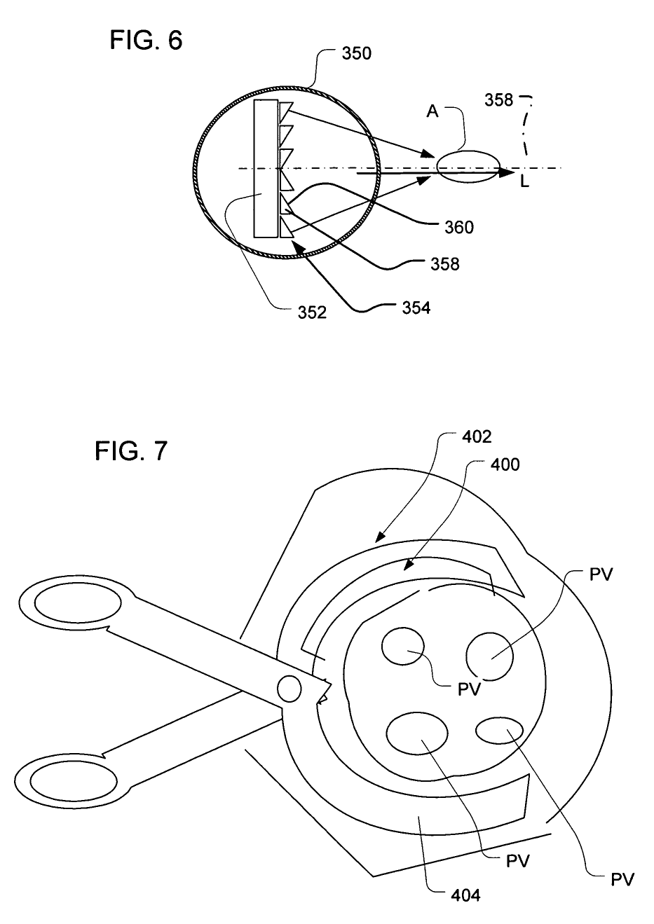

Apparatus according to a further embodiment of the invention includes a hollow elongated probe 350 and an elongated emitter 352 (FIG. 6) having an emitting surface facing in the lateral direction. In FIG. 6, as in FIGS. 2-5 discussed above, the probe and emitter are seen in sectional view. Here again, the emitter extends lengthwise along the probe within the emitting region, i.e., into and out of the plane of the drawing in FIG. 6. Here again, the emitter may include numerous emitting elements, each extending for only a small portion of the overall length of the emitter. A lens 354 formed from a solid material having acoustic velocity different from than that of water, but an acoustic impedance close to that of water as, for example, polystyrene or other polymer, is positioned over the emitting surface. The lens is a multi-element lens including an array of individual refractive elements 356. The lens also extends lengthwise, into and out of the plane of the drawing. The elements of the lens are shaped so that ultrasonic waves propagating from emitting element 352 in lateral direction L will be refracted toward the medial plane 358 and hence focused into ablation region A. The lens may be a Fresnel lens with each element having a surface 360 sloping so that the vector normal to such surface points toward the medial plane 358, as shown schematically in FIG. 6. Alternatively, the lens may have individual elements of other configurations arranged so that the delays imparted by the elements cause ultrasonic waves passing out of the lens to mutually reinforce one another at the medial plane. For example, where the emitting surface of element 352 emits waves in phase with one another, the thicknesses of the various elements may be selected so that the total propagation delay for ultrasonic waves traveling through each element to a theoretical line 362 extending in the medial plane is equal. The individual elements of a multi-element lens can be thin, on the order of a few millimeters and therefore the multi-element lens may be flexible to facilitate insertion and positioning of the device. Alternatively, the multi-element lens may be made in sections corresponding to the individual emitting elements.

In the embodiments above, the probe is shown as a simple elongated element which is introduced into the epicardial space, inside the pericardium, by a tubular introducer. However, the probe need not be arranged for introduction through a tubular introducer. For example, where the procedure is performed using an open-chest surgical approach, the probe may be introduced directly through a hole in the pericardium, without the use of a separate introducer. In such cases, the probe body may be a rigid structure having a predetermined curvature. In other arrangements, the probe body may include conventional features for deforming a catheter or other probe to a desired shape as, for example, nitinol or other shape memory alloy elements which assume the desired shape when exposed to body temperature. The probe may also include “steering” elements of the types commonly used in steerable catheters as, for example, pull wires for selectively bending the probe into a desired shape or while the same is in place within the patient's body. The probe also may be provided with sensors for detecting the position of the probe relative to an external frame of reference established, for example, by a magnetic or optical position-locating system similar to those used in stereotactic surgery. Also, the probe may be formed from radio opaque materials or other materials detectable in fluoroscopic images. In other arrangements, the fluids used to fill the probe may be radio opaque. For example, the liquid used in a chamber of a refractive embodiment (e.g., chamber 230; FIG. 3) may incorporate a radio opaque dye. Alternatively or additionally, the liquid used in the first chamber 28 of the reflecting probe discussed above with reference to FIGS. 1 and 2 carry a radio opaque contrast medium.

Apparatus according to a further embodiment of the invention (FIG. 6) incorporates an elongated focused ultrasound element 400 which may include any of the transducer arrays and focusing arrangements discussed above with reference to the probes of FIGS. 1-5. This ultrasonic application device 400, however, is not mounted on the body of a rod-like probe, but instead is mounted on a blade 402 of a forceps-like device. Blade 400 is pivotally connected to an opposing blade 404. The forceps-like device may be engaged around the outside of the heart, as by opening the pericardium surgically and advancing the blades into the opening. Once positioned, the blades may be moved towards one another so as to engage the elongated ultrasonic device and focusing arrangement with the epicardial surface of the heart. Here again, the ultrasonic ablation device, with its focusing arrangement, provides highly concentrated ultrasound along a path corresponding to the length of the ablation device.

As these and other variations and combinations of the features discussed above can be utilized without departing from the present invention, the foregoing description of the preferred embodiments should be taken by way of illustration rather than by way of limitation of the invention as defined by the claims.

1. Apparatus for treating cardiac arrhythmias comprising:
(a) an ultrasonic ablation device including an ultrasonic emitter and means for focusing ultrasonic waves emitted by said emitter into an ablation region; and
(b) means for positioning said ablation device outside of the heart adjacent the epicardial surface of the heart so that said ablation region is disposed within the wall of the heart.

2. Apparatus as claimed in claim 1 wherein said means for positioning includes an elongated probe having a lengthwise direction, said ultrasonic ablation device being carried by said probe, said means for focusing being operative to focus the energy from said emitter into an elongated ablation region extending generally parallel to said lengthwise direction of said probe and offset from the probe in a lateral direction transverse to said lengthwise direction.

3. Apparatus as claimed in claim 2 wherein said ultrasonic emitter includes one or more ultrasonic emitting elements mounted to said probe, said one or more emitting elements defining an emitting surface extending in said lengthwise direction.

4. Apparatus as claimed in claim 3 wherein said focusing means includes a lens carried on said probe, said lens being elongated in said lengthwise direction, said emitting surface facing toward said lens so that ultrasonic waves emanating from said emitting surface are directed into said lens.

5. Apparatus as claimed in claim 4 wherein said lens is inflatable.

6. Apparatus as claimed in claim 4 wherein said lens is a multi-element lens.

7. Apparatus as claimed in claim 3 wherein said focusing means includes a concave reflective surface facing in the lateral direction.

8. Apparatus as claimed in claim 7 wherein said probe includes structure defining first and second chambers and a common wall separating said first and second chambers, said emitter being disposed in said second chamber, and wherein said probe further includes a connector for connecting said first chamber to a source of a liquid and connecting said second chamber to a source of a gas, whereby said common wall forms said concave reflective surface.

9. Apparatus as claimed in claim 3 wherein said focusing means includes one or more structures carried by said probe defining a pair of reflective surfaces disposed bounding a slot extending lengthwise along said probe, said slot having an entry opening adjacent said emitting surface and an exit opening remote from said emitting surface, said reflective surfaces converging with one another from said entry opening to said exit opening.

10. Apparatus as claimed in claim 9 wherein said structures defining said reflective surfaces include one or more walls bounding one or more chambers, the apparatus further including a gas inlet communicating with said one or more chambers.

11. Apparatus as claimed in claim 2 wherein said emitting surface includes surface regions positioned on said probe so that vectors normal to said surface regions converge with one another, said focusing means including said surface regions.

12. Apparatus as claimed in claim 11 wherein said surface regions include surface regions disposed on opposite sides of a medial plane extending in said lengthwise direction and said lateral direction, and wherein said surface regions include regions disposed on opposite sides of said medial plane having normal vectors sloping towards said medial plane.

13. A method of treating cardiac arrhythmias comprising the steps of:
(a) positioning an elongated ultrasonic emitter outside of the heart but adjacent the heart so that the emitter extends along an elongated path on the heart wall; and
(b) actuating the ultrasonic emitter to emit ultrasonic waves while directing such ultrasonic waves into an ablation region extending along said path within the heart wall and focusing such ultrasonic waves into the ablation region.
